(19) United States
(12) Patent Application Publication
(10) Pub. No.: US 2026/0269029 A1
(43) Pub. Date: Sep. 10, 2026

(54) HEALTH INFORMATION PROCESSING DEVICE, HEALTH INFORMATION PROCESSING METHOD, AND STORAGE MEDIUM

(71) Applicant: NEC Corporation, Tokyo (JP)

(72) Inventor: Masanori TSUJIKAWA, Tokyo (JP)

(73) Assignee: NEC Corporation, Tokyo (JP)

(21) Appl. No.: 19/163,533

(22) PCT Filed: Mar. 24, 2023
(86) PCT No.: PCT/JP2023/011862

§ 371 Date: Sep. 9, 2025

(51) Int. Cl. G16H 10/60

(52) U.S. Cl. CPC G16H 10/60

(57) ABSTRACT

A health information processing device mainly includes a summarization degree setting means 16X and a summary generation means 17X. The summarization degree setting means 16X is configured to set, based on a relationship of injury/disease between a plurality of treatment periods of a subject, a degree of summarization of medical treatment records for each of the plurality of treatment periods. The summary generation means 17X is configured to generate a summary of the medical treatment records in a target period including the plurality of treatment periods based on the medical treatment records and the degree of summarization.

### TECHNICAL FIELD

The present disclosure relates to a technical field of a health information processing device, a health information processing method, and a storage medium that perform processing related to health information.

### BACKGROUND

Among various types of work performed by a medical staff, work of recording a medical practice performed by the medical staff on a patient is essential for sharing patient information with a related medical staff, proving that an objectively appropriate medical practice has been performed, and the like. On the other hand, the work related to the medical treatment record has a large burden on the medical staff. As a technology related to assistance of the work related to the medical treatment record, PTL 1 discloses a technology of generating a summary from medical treatment records by a medical staff.

### CITATION LIST

### Patent Literature

PTL 1: JP 2020-38602 A

### SUMMARY

### Problem to be Solved

In a case where a summary of medical treatment records in a target period including a plurality of treatment periods is generated, when the medical treatment records included in the target period are simply summarized by the same degree of compression, there is a possibility that a summary accurately and briefly representing a state of an object patient is not generated.

In view of the above-described problem, an object of the present disclosure is to provide a health information processing device, a health information processing method, and a storage medium capable of suitably generating a summary of medical treatment records in a target period.

### Means for Solving the Problem

One mode of the health information processing device is a health information processing device including:
a summarization degree setting means for setting, based on a relationship of injury/disease between a plurality of treatment periods of a subject, a degree of summarization of medical treatment records for each of the plurality of treatment periods; and
a summary generation means for generating a summary of the medical treatment records in a target period including the plurality of treatment periods based on the medical treatment records and the degree of summarization.

One mode of the health information processing method is a health information processing method executed by a computer, includes:
setting, based on a relationship of injury/disease between a plurality of treatment periods of a subject, a degree of summarization of medical treatment records for each of the plurality of treatment periods; and
generating a summary of the medical treatment records in a target period including the plurality of treatment periods based on the medical treatment records and the degree of summarization.

One mode of the storage medium is a storage medium storing a program executed by a computer, the program causing the computer to:
set, based on a relationship of injury/disease between a plurality of treatment periods of a subject, a degree of summarization of medical treatment records for each of the plurality of treatment periods; and
generate a summary of the medical treatment records in a target period including the plurality of treatment periods based on the medical treatment records and the degree of summarization.

### Effect

An example advantage according to the present invention is to suitably generate a summary of medical treatment records in a target period.

### BRIEF DESCRIPTION OF THE DRAWINGS

FIG. 1 illustrates a schematic configuration of a health information processing system according to a first example embodiment.

FIG. 2 is an example of a data structure of a medical treatment record in a medical treatment record storage unit.

FIG. 3 illustrates a hardware configuration of a health information processing device.

FIG. 4 is an example of functional blocks of a processor of the health information processing device.

FIG. 5 is a diagram illustrating a relationship between treatment periods (first treatment period to third treatment period) of an object patient and examples of set target periods (a target period a and a target period ß).

FIG. 6 is an example of a flowchart executed by the health information processing device in the first example embodiment.

FIG. 7 illustrates a schematic configuration of a health information processing system according to a second example embodiment.

FIG. 8 is an example of a data structure of summary information in a summary information storage unit.

FIG. 9 illustrates a schematic configuration of a health information processing system in a third example embodiment.

FIG. 10 is a block diagram of a health information processing device in a fourth example embodiment.

FIG. 11 is an example of a flowchart executed by the health information processing device in the fourth example embodiment.

### EXAMPLE EMBODIMENTS

Hereinafter, example embodiments of a health information processing device, a health information processing method, and a storage medium will be described with reference to the drawings.

### First Example Embodiment

### (1) System Configuration

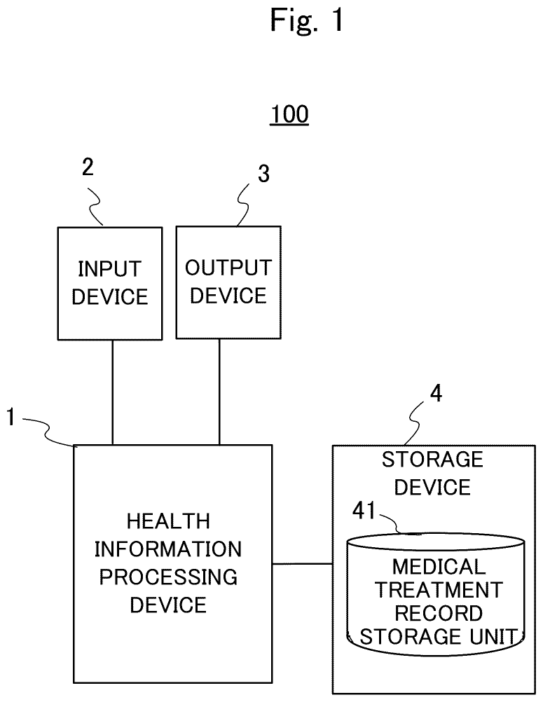

FIG. 1 illustrates a schematic configuration of a health information processing system 100 according to a first example embodiment. The health information processing system 100 generates a summary of medical treatment records of a patient in a specified period.

Hereinafter, the “medical treatment records” refer to various records created in a process of medical treatment for states (a physical condition, a condition of a disease, treatment to be applied, and the like) of the patient, and includes a medical record (clinical record), a prescription, an operation record, a nursing record, an examination observation record, an X-ray photograph, a referral, a summary of a medical treatment progress during hospitalization related to the discharged patient, and the like. Hereinafter, unless otherwise stated, the medical treatment records are assumed to refer to electronic data in which the above-described various records are electronically stored. The “summary of medical treatment records” is assumed to include at least a sentence (summary sentence) obtained by summarizing sentences included in the medical treatment records.

The health information processing system 100 mainly includes a health information processing device 1, an input device 2, an output device 3, and a storage device 4.

The health information processing device 1 performs processing related to generation of a summary of medical treatment records. The health information processing device 1 performs data communication with the input device 2, the output device 3, and the storage device 4 via a communication network or by wireless or wired direct communication. Based on an input signal or the like supplied from the input device 2, for example, the health information processing device 1 specifies a patient (also referred to as an “object patient”) whose medical treatment records are to be summarized, a period (also referred to as an “target period”) in which medical treatment records are to be summarized, and the like. Here, it is assumed that the target period includes a plurality of treatment periods of injury/disease suffered by the object patient. The health information processing device 1 generates a summary (also referred to as a “second medical treatment summary”) of the medical treatment records in the target period based on a summary (also referred to as a “first medical treatment summary”) of the medical treatment records for each treatment period.

The health information processing device 1 also generates an output signal, and supplies the generated output signal to the output device 3. The output signal in this case is, for example, display information (audio information may be included) for receiving an input necessary for generating the second medical treatment summary or a correction input of the generated second medical treatment summary. The health information processing device 1 may store the generated information related to the second medical treatment summary in the storage device 4, or may transmit the generated information related to the second medical treatment summary to an external device (not illustrated). The generated second medical treatment summary is used to create, for example, a referral, a medical certificate, a discharge summary, or another medical document that requires description of a summary of medical treatment records, which is created by a medical staff.

The input device 2 is an interface that receives a manual input (external input) of information by a user. The input device 2 may be, for example, various user input interfaces such as a touch panel, a button, a keyboard, a mouse, and an audio input device. The input device 2 supplies a generated input signal to the health information processing device 1. The output device 3 outputs predetermined information based on an output signal supplied from the health information processing device 1. The output device 3 is, for example, a display, a projector, a speaker, or a printer.

The storage device 4 is a memory that stores various types of information required for processing to be executed by the health information processing device 1. The storage device 4 may be an external storage device such as a hard disk connected to or incorporated in the health information processing device 1, or may be a storage medium such as a flash memory. The storage device 4 may be a server device that performs data communication with the health information processing device 1. The storage device 4 may include a plurality of devices. At least a part of the information stored in the storage device 4 may be stored by the health information processing device 1.

The storage device 4 functionally mainly includes a medical treatment record storage unit 41 that stores medical treatment records of a patient.

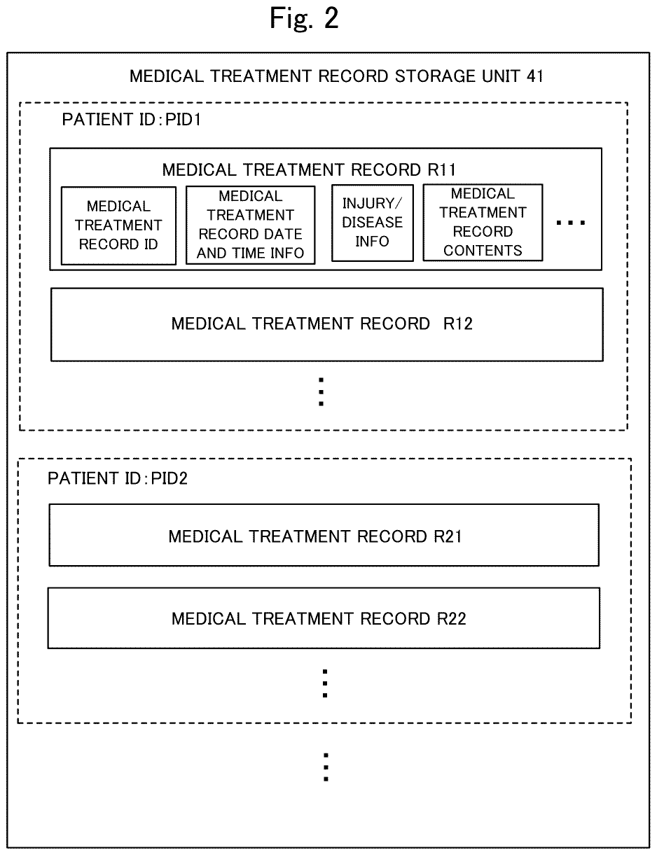

FIG. 2 is an example of a data structure of the medical treatment record in the medical treatment record storage unit 41. The medical treatment record storage unit 41 stores, for example, a medical treatment record created for each medical treatment in association with patient identification information (also referred to as a “patient ID”) for identifying a patient who has received the medical treatment. In the example of FIG. 2, the medical treatment record storage unit 41 stores a medical treatment record “R11”, a medical treatment record “R12”, and the like of a patient indicated by a patient ID “PID1” in association with the patient ID “PID1”, and stores a medical treatment record “R21”, a medical treatment record “R22”, and the like of a patient indicated by a patient ID “PID2” in association with the patient ID “PID2”.

Each record of the medical treatment record includes a medical treatment record ID as identification information of the medical treatment record, medical treatment record date and time information indicating a date and time when the medical treatment record is created (or the medical treatment is performed), injury/disease information indicating injury/disease to be treated, and medical treatment record contents indicating contents of the medical treatment record (for example, a clinical record) related to a state of the patient. The health information processing device 1 in the first example embodiment generates a first medical treatment summary and a second medical treatment summary obtained by summarizing sentences indicated by the medical treatment record contents.

The data structure of each record of the medical treatment record is not limited to that illustrated in FIG. 2. For example, information (including text information) related to the injury/disease information may be incorporated in the medical treatment record contents. In this case, the injury/disease may be specified by text analysis of the medical treatment record contents. The medical treatment record stored in the medical treatment record storage unit 41 may be a collection of the records for each treatment period of the same patient as one record. In this case, the medical treatment record date and time information may be information indicating a treatment period.

The configuration of the health information processing system 100 illustrated in FIG. 1 is an example, and various changes may be made to the configuration. For example, the input device 2 and the output device 3 may be integrally configured. In this case, the input device 2 and the output device 3 may be configured as a tablet terminal (including a smartphone) integrated with or separated from the health information processing device 1. The health information processing device 1 may include a plurality of devices. In this case, the plurality of devices constituting the health information processing device 1 exchanges information necessary for executing processing allocated in advance between the plurality of devices. In this case, the health information processing device 1 functions as a system.

### (2) Hardware Configuration

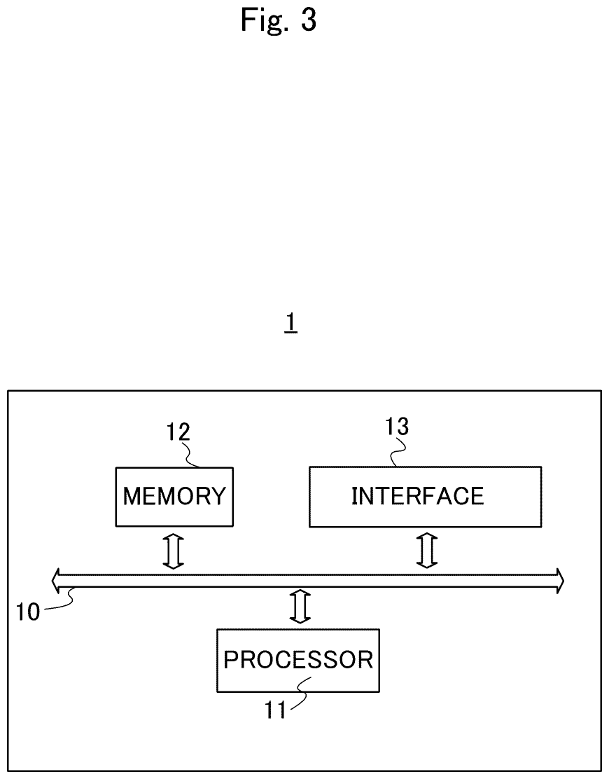

FIG. 3 illustrates a hardware configuration of the health information processing device 1. The health information processing device 1 includes, as hardware, a processor 11, a memory 12, and an interface 13. The processor 11, the memory 12, and the interface 13 are connected to each other via a data bus 10.

The processor 11 functions as a controller (arithmetic device) that takes overall control of the health information processing device 1 by executing a program stored in the memory 12. The processor 11 is, for example, a processor such as a central processing unit (CPU), a graphics processing unit (GPU), or a tensor processing unit (TPU). The processor 11 may include a plurality of processors. The processor 11 is an example of a computer.

The memory 12 includes various volatile memories and nonvolatile memories, such as a random access memory (RAM), a read only memory (ROM), and a flash memory. The memory 12 stores a program for executing processing executed by the health information processing device 1. A part of information stored in the memory 12 may be stored by one or a plurality of external storage devices capable of communicating with the health information processing device 1, or may be stored by a storage medium attachable to and detachable from the health information processing device 1.

The interface 13 is an interface for electrically connecting the health information processing device 1 and another device. These interfaces may be a wireless interface such as a network adapter for wirelessly transmitting and receiving data to and from the another device, or may be a hardware interface for connecting to the another device by a cable or the like.

The hardware configuration of the health information processing device 1 is not limited to the configuration illustrated in FIG. 3. For example, the health information processing device 1 may include at least one of the input device 2 and the output device 3.

### (3) Generation of Second Medical Treatment Summary

Next, processing related to the generation of a second medical treatment summary executed by the health information processing device 1 will be described. Schematically, the health information processing device 1 determines a degree of summarization (also referred to as a “summarization degree”) of medical treatment records for each treatment period based on a relationship of injury/disease between treatment periods, and generates a second medical treatment summary in a target period by using a first medical treatment summary generated based on the determined summarization degree. As a result, the health information processing device 1 generates the second medical treatment summary obtained by accurately summarizing the medical treatment records in the target period. Hereinafter, it is assumed that a higher value of the summarization degree indicates that a summarized sentence is a brief sentence (that is, a sentence having a higher degree of compression).

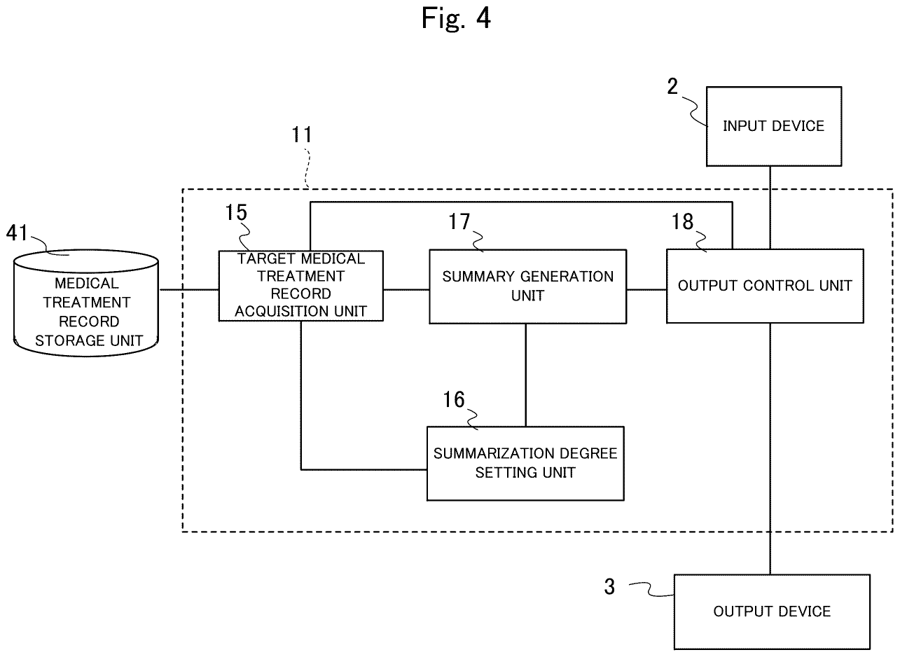

FIG. 4 is an example of functional blocks of the processor 11 of the health information processing device 1. The processor 11 of the health information processing device 1 functionally includes a target medical treatment record acquisition unit 15, a summarization degree setting unit 16, a summary generation unit 17, and an output control unit 18. Here, while blocks that exchange data with each other are connected by a solid line, a combination of the blocks that exchange data with each other is not limited to this. The same applies to diagrams of other functional blocks described later.

The target medical treatment record acquisition unit 15 acquires medical treatment records related to a specified target period and object patient (also referred to as “target medical treatment records”) from the medical treatment record storage unit 41 via the interface 13. In this case, for example, the output control unit 18 receives a user input specifying the target period and the object patient by the input device 2, and supplies an input signal generated by the input device 2 to the target medical treatment record acquisition unit 15. The target medical treatment record acquisition unit 15 extracts the related target medical treatment records from the medical treatment record storage unit 41 based on the target period and the object patient indicated by the input signal. In this case, the target medical treatment record acquisition unit 15 extracts the medical treatment records that include medical treatment record date and time information indicating a date and time included in the target period and are associated with a patient ID of the object patient from the medical treatment record storage unit 41 as the target medical treatment records. The target medical treatment record acquisition unit 15 supplies the target medical treatment records to the summarization degree setting unit 16 and the summary generation unit 17.

The summarization degree setting unit 16 specifies injury/disease of the object patient for each treatment period based on the target medical treatment records supplied from the target medical treatment record acquisition unit 15, and sets a summarization degree of a first medical treatment summary related to each treatment period based on a determination result of a relationship of the specified injury/disease.

In this case, the summarization degree setting unit 16 first classifies the plurality of time-series target medical treatment records supplied from the target medical treatment record acquisition unit 15 as one group of time-series medical treatment records related to the same injury/disease. The summarization degree setting unit 16 then specifies a period indicated by medical treatment record dates and times for each group (group) of the target medical treatment records formed by the above-described classification as each treatment period of the object patient. In a case where a record of the medical treatment records is generated for each treatment period, the summarization degree setting unit 16 specifies the treatment period based on the medical treatment record date and time information of the medical treatment records.

The summarization degree setting unit 16 regards a last treatment period in the target period (that is, the latest treatment period) as a treatment period as a reference in determination of the relationship (also referred to as a “reference treatment period”), and determines a degree of relevance (also referred to as a “relevance degree”) between injury/disease in the latest treatment period and injury/disease in each treatment period. In this case, for example, information indicating a relationship between a candidate of a set of injury/disease for which a relevance degree is determined and a relevance degree to be set for each candidate is stored in the storage device 4, the memory 12, or the like, and the summarization degree setting unit 16 determines, by referring to the information, the relevance degree between the injury/disease in the reference treatment period and the injury/disease in each treatment period. Here, the relevance degree related to the reference treatment period is set to be a maximum value.

The summarization degree setting unit 16 sets the summarization degree higher for a treatment period with a lower relevance degree. In this case, for example, information indicating a relationship between a candidate of an assumed relevance degree and a summarization degree to be set for each candidate is stored in the storage device 4, the memory 12, or the like, and the summarization degree setting unit 16 determines, by referring to the information, the summarization degree from the determined relevance degree for each treatment period. Since the relevance degree related to the reference treatment period is the maximum value, the summarization degree related to the reference treatment period is a minimum value (that is, a value indicating that the degree of summarization is the lowest).

A method of setting the summarization degree is not limited to the above-described example. For example, in a case where information indicating a relationship between the candidate of the set of injury/disease for which the relevance degree is determined and the summarization degree to be set for each candidate is stored in the storage device 4, the memory 12, or the like, the summarization degree setting unit 16 may directly determine, by referring to the information, a summarization degree related to an optional treatment period from the injury/disease in the latest treatment period and injury/disease in the optional treatment period.

In another example, learned parameters of an inference model that has learned the relationship between the set of injury/disease and the summarization degree to be set may be stored in the storage device 4, the memory 12, or the like. In this case, the summarization degree setting unit 16 inputs a set of the injury/disease in the latest treatment period and the injury/disease in the optional treatment period to the inference model configured based on the parameters, and acquires a summarization degree output by the inference model as the summarization degree related to the optional treatment period.

In still another example, in the determination of the relevance degree, in a case where description in which the relationship of the injury/disease between the treatment periods is clearly described in the target medical treatment records is detected, the summarization degree setting unit 16 may determine the above-described relevance degree based on the description. In this case, for example, in a case where description of the target medical treatment record clearly described as being related to injury/disease related to a specific treatment period is detected, the summarization degree setting unit 16 sets, to a maximum value, the relevance degree of the injury/disease between the treatment period of the target medical treatment record having the above-described description and the above-described specific treatment period.

The summary generation unit 17 generates a first medical treatment summary for each treatment period based on the target medical treatment records supplied from the target medical treatment record acquisition unit 15 and the summarization degree for each treatment period supplied from the summarization degree setting unit 16. A method of generating the first medical treatment summary based on the summarization degree will be described later. The summary generation unit 17 generates a second medical treatment summary based on the first medical treatment summary generated for each treatment period. In this case, for example, the summary generation unit 17 generates the second medical treatment summary indicating a sentence obtained by connecting sentences indicated by the first medical treatment summary according to a time series of the treatment period. The summary generation unit 17 supplies the generated second medical treatment summary to the output control unit 18.

The output control unit 18 causes the output device 3 to display an input screen that receives the input for specifying the target period and the object patient, and receives, by the input device 2, the user input for specifying the target period and the object patient on the input screen. The output control unit 18 supplies the input signal specifying the target period and the object patient input by the input device 2 to the target medical treatment record acquisition unit 15. The output control unit 18 also causes the output device 3 to display the second medical treatment summary generated by the summary generation unit 17, and receives a correction input of the second medical treatment summary by the input device 2. In this case, for example, the output control unit 18 displays the sentence indicated by the second medical treatment summary in an input field in which text editing is possible. After that, the output control unit 18 may perform processing of storing the second medical treatment summary generated by the summary generation unit 17 or the second medical treatment summary corrected by the received correction input in the storage device 4, the memory 12, or the like, transmitting the second medical treatment summary to another device, or printing the second medical treatment summary by a printer.

In a case where the second medical treatment summary is applied to a medical document (for example, a referral, a medical certificate, a discharge summary, and the like), the output control unit 18 may further correct the second medical treatment summary in such a way that the second medical treatment summary falls within an entry frame for filling in the second medical treatment summary in the medical document. For example, in a case where the number of characters that can be input in the above entry frame is set, the second medical treatment summary is further summarized in such a way as to be within the number of characters. In this case, the output control unit 18 may summarize the second medical treatment summary by using an optional summarization method. The output control unit 18 is an example of “correction input reception means” and “correction means”.

The components of the target medical treatment record acquisition unit 15, the summarization degree setting unit 16, the summary generation unit 17, and the output control unit 18 described in FIG. 4 can be achieved by, for example, the processor 11 executing a program. Each component may also be achieved by recording a necessary program in an optional nonvolatile storage medium and installing the program as necessary. At least a part of these components is not limited to be achieved by software by the program, and may be achieved by a combination of any of hardware, firmware, and software, or the like. At least a part of these components may be achieved by using, for example, a user-programmable integrated circuit such as a field-programmable gate array (FPGA) or a microcontroller. In this case, a program including the above components may be achieved by using the integrated circuit. At least a part of the components may include an application specific standard produce (ASSP), an application specific integrated circuit (ASIC), or a quantum processor (quantum computer control chip). In this manner, the components may be achieved by various types of hardware. The same applies to other example embodiments described later. These components may also be achieved by, for example, cooperation of a plurality of computers by using a cloud computing technology or the like. The same applies to other example embodiments described later.

### (4) Generation of First Medical Treatment Summary Based on Summarization Degree

Next, the method of generating a first medical treatment summary based on the summarization degree will be described.

First, the summary generation unit 17 groups target medical treatment records for each treatment period, and performs text analysis of sentences included in medical treatment record contents of the target medical treatment records for each group. Here, examples of the “text analysis” include morphological analysis, syntax analysis, context analysis, time information analysis, factuality analysis, named entity recognition, and semantic analysis.

The summary generation unit 17 then generates a first medical treatment summary for each treatment period based on a text analysis result and a summarization degree. For example, the summary generation unit 17 determines, for each sentence (that may be for each paragraph, and the same applies hereinafter), whether to leave or delete the sentence as a sentence to be included in the first medical treatment summary based on importance degrees of words extracted from the target medical treatment records by the text analysis, and determines the above-described determination criterion based on the summarization degree. The above-described determination criterion is, for example, a threshold to be compared with the importance degrees of the extracted words, and is set to be a stricter criterion as a level of the summarization degree is higher. For example, information (for example, an expression or table information) indicating a correspondence relationship between the summarization degree and the determination criterion is stored in advance in the storage device 4, the memory 12, or the like, and the summary generation unit 17 determines, with reference to this information, the determination criterion from the summarization degree. The importance degree of the word may be determined based on a category classified by the named entity recognition or the like. For example, in a case where information indicating a correspondence relationship between the category and the importance degree is stored in advance in the storage device 4, the memory 12, or the like, the summary generation unit 17 determines, with reference to this information, the importance degree for each extracted word from the classified category.

Next, a specific example of the first medical treatment summary according to the summarization degree will be described. Here, as an example, it is assumed that the summarization degree is divided into three stages of a lowest “level 1”, a highest “level 3”, and a “level 2” intermediate between the level 1 and the level 3. The first medical treatment summaries based on the summarization degrees of the level 1 to the level 3 are as follows.

### Level 1

“Referred to our department for the first time on Sep. 29, 2020. An examination for make a definitive diagnosis required time. Diagnosed in November 2020 as oropharyngeal adenoid cystic carcinoma with multiple lung metastases. Since there are no obvious symptoms and it is a tissue type expected to progress slowly, it was decided that watchful waiting is used. Currently being subjected to watchful waiting with no treatment on an outpatient basis. At the time of consultation on Oct. 12, 2021, there was no obvious increase, but symptoms such as taking time to eat and difficulty in speaking appeared. A treatment plan will be considered after regular consultation.”

### Level 2

“Referred to our department for the first time on Sep. 29, 2020. Diagnosed in November 2020 as oropharyngeal adenoid cystic carcinoma with multiple lung metastases. Currently being subjected to watchful waiting with no treatment on an outpatient basis. At the time of consultation on Oct. 12, 2021, there was no obvious increase, but symptoms such as taking time to eat and difficulty in speaking appeared.”

### Level 3

“Diagnosed in November 2020 as oropharyngeal adenoid cystic carcinoma with multiple lung metastases. Currently being subjected to watchful waiting with no treatment on an outpatient basis. At the time of consultation on Oct. 12, 2021, there was no obvious increase.”

In this specific example, it is determined whether to leave each sentence as a summary sentence based on an importance degree or the like of a word extracted by text analysis or the like, and a determination criterion (for example, a threshold for the above-described importance degree) as to whether to leave the sentence as the summary sentence is a stricter criterion as a level of a summarization degree is higher.

For example, only sentences related to a treatment method, a disease name, and a date and time are left in the first medical treatment summary having the summarization degree of the level 3 in the above-described specific example, and as a result, the sentences are briefer (that is, have a higher degree of compression) than the summary sentences of the level 1 and the level 2. The first medical treatment summary having the summarization degree of the level 1 may directly include the sentences included in the medical treatment record contents of the target medical treatment records (that is, the sentences are not deleted by summarization).

The first medical treatment summary according to the importance degree may be generated by an optional method, not limited to the above example. For example, the summary generation unit 17 may use a model that generates a summary of sentences according to a summarization degree in a case where the summarization degree and the sentences are input. In this case, parameters of the above-described model are stored in advance in the storage device 4, the memory 12, or the like, and the summary generation unit 17 inputs a summarization degree and sentences of target medical treatment records for each group to the model configured using the parameters. The summary generation unit 17 then acquires a summary output by the model as the first medical treatment summary.

### (5) Specific Examples of Treatment Period and Target Period

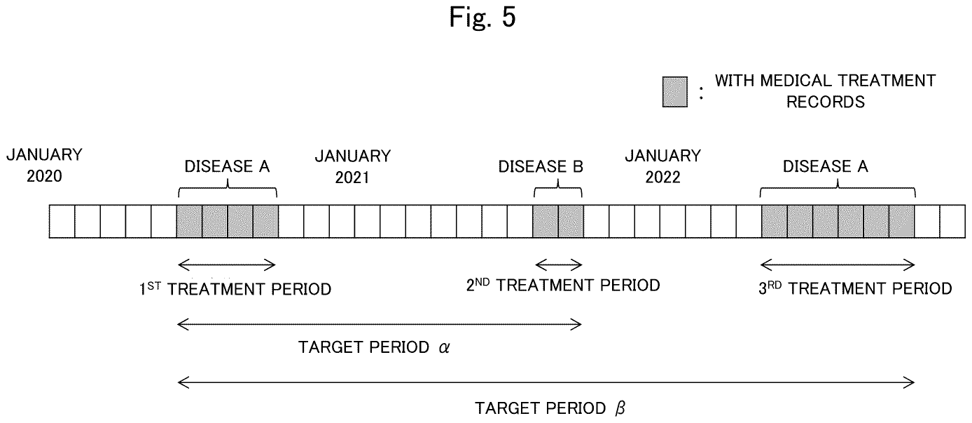

FIG. 5 is a diagram illustrating a relationship between treatment periods (a first treatment period to a third treatment period) of an object patient and examples of set target periods (a target period a and a target period B). In FIG. 5, a period from January 2020 is represented by a column of slots each of which is related to a period of one month, and slots related to months in which medical treatment records are created are filled. Here, it is assumed that the object patient has been treated for a disease A in the first treatment period, the object patient has been treated for a disease B in the second treatment period, and the object patient has been treated for the disease A in the third treatment period.

As a first example, a case will be described where the target period a including the first treatment period and the second treatment period is set. In this case, the health information processing device 1 generates a first medical treatment summary related to the first treatment period and a first medical treatment summary related to the second treatment period, and then generates a second medical treatment summary related to the target period a based on these first medical treatment summaries. At this time, the health information processing device 1 in the present example embodiment regards the second treatment period related to a last treatment period in the target period α as a reference treatment period, and determines relevance degrees of injury/disease between the disease B suffered by the object patient in the reference treatment period (that is, the second treatment period) and the diseases (the disease A and the disease B) suffered by the object patient in the treatment periods (that is, the first treatment period and the second treatment period). The health information processing device 1 then determines a summarization degree related to the first treatment period according to the relevance degree between the disease B in the reference treatment period (that is, the second treatment period) and the disease A in the first treatment period. Since the relevance degree related to the second treatment period as the reference treatment period is a maximum value, the summarization degree related to the second treatment period is a minimum value.

In this manner, in the first example, the health information processing device 1 can determine the summarization degree (that is, the degree of compression) of the medical treatment records in the first treatment period included in the second medical treatment summary related to the target period a according to the relationship between the disease B in the latest second treatment period and the disease A in the first treatment period.

Next, as a second example, a case will be described where the target period β including the first treatment period, the second treatment period, and the third treatment period is set. In this case, the health information processing device 1 generates a first medical treatment summary related to the first treatment period, a first medical treatment summary related to the second treatment period, and a first medical treatment summary related to the third treatment period, and then generates a second medical treatment summary related to the target period β based on these first medical treatment summaries. At this time, the health information processing device 1 in the present example embodiment regards the third treatment period related to a last treatment period in the target period β as a reference treatment period, and determines relevance degrees of injury/disease between the disease A suffered by the object patient in the reference treatment period (that is, the third treatment period) and the diseases suffered by the object patient in the treatment periods (that is, the first treatment period to the third treatment period). In this case, since the first treatment period and the third treatment period relate to the same disease A as that of the reference treatment period (that is, the third treatment period), the relevance degree in these periods is a maximum value, and a summarization degree is a minimum value. A summarization degree related to the second treatment period is determined according to a relevance degree between the disease A in the reference treatment period and the disease B in the second treatment period.

In this manner, in the second example, the health information processing device 1 can determine the summarization degree (that is, the degree of compression) of the medical treatment records in the past first treatment period and second treatment period included in the second medical treatment summary related to the target period β according to the relationships between the disease A in the latest third treatment period and the diseases in the treatment periods.

### (6) Processing Flow

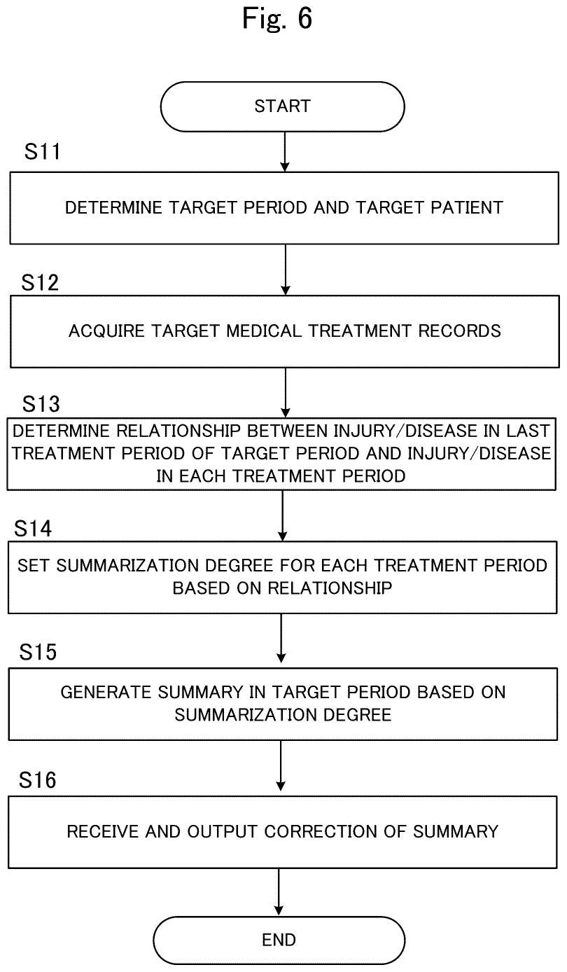

FIG. 6 is an example of a flowchart executed by the health information processing device 1 in the first example embodiment.

First, the health information processing device 1 determines a target period and an object patient for which summarization of medical treatment records is performed (step S11). The health information processing device 1 determines the target period and the object patient based on, for example, a user input from the input device 2. In a case where the target period is not specified, for example, the health information processing device 1 may determine, as the target periods, all periods (or may be another predetermined period) in which the medical treatment records for the object patient exist. The health information processing device 1 then acquires the target medical treatment records related to the target period and the object patient determined in step S11 from the medical treatment record storage unit 41 (step S12).

Next, the health information processing device 1 determines a relationship between injury/disease in a last treatment period of the target period (that is, a reference treatment period) and injury/disease in each treatment period included in the target period (step S13). As a result, the health information processing device 1 determines the relationship for each treatment period. The health information processing device 1 then sets a summarization degree for each treatment period based on the relationship determined for each treatment period in step S13 (step S14). The health information processing device 1 then generates a summary of the medical treatment records of the object patient in the target period (that is, a second medical treatment summary) based on the summarization degree set in step S14 (step S15). In this case, the health information processing device 1 generates a first medical treatment summary obtained by summarizing the medical treatment records for each treatment period based on the summarization degree set for each treatment period, and generates the second medical treatment summary obtained by combining the generated first medical treatment summaries.

Next, the health information processing device 1 receives correction of the second medical treatment summary, and outputs the second medical treatment summary reflecting the correction (step S16). In this case, for example, the health information processing device 1 displays the second medical treatment summary by the output device 3, receives the correction input of the second medical treatment summary by the input device 2, and corrects the second medical treatment summary in such a way as to fall within an entry frame of a medical document to be applied. The health information processing device 1 may store the finally generated second medical treatment summary in the storage device 4, the memory 12, or the like, may transmit the second medical treatment summary to another device, or may print the second medical treatment summary by a printer.

### second Example Embodiment

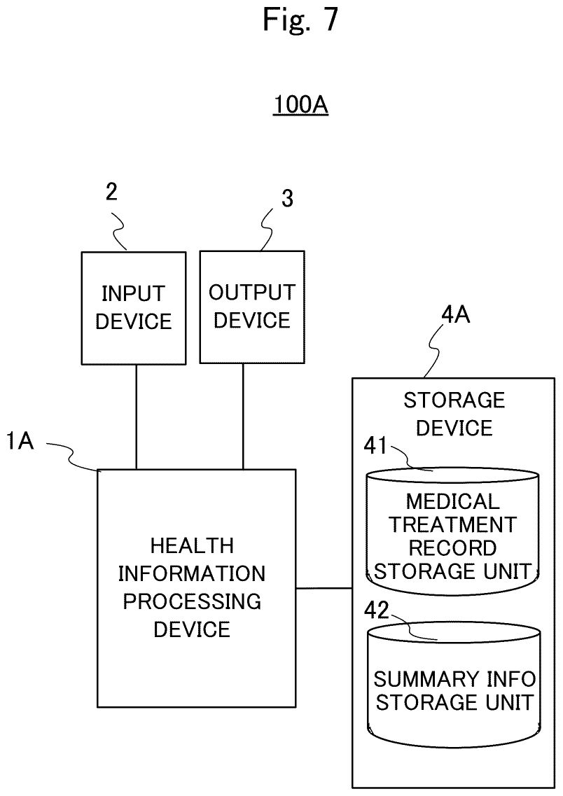

FIG. 7 illustrates a schematic configuration of a health information processing system 100A according to a second example embodiment. The health information processing system 100A according to the second example embodiment is different from the health information processing system 100 of the first example embodiment in that a summary of medical treatment records for each treatment period is stored in advance in a storage device 4A. Hereinafter, the same components as those of the first example embodiment are appropriately denoted by the same reference signs, and description thereof will be omitted.

The storage device 4A functionally includes a medical treatment record storage unit 41 and a summary information storage unit 42. The summary information storage unit 42 stores information (also referred to as “summary information”) indicating a summary generated for each treatment period of injury/disease suffered by a patient.

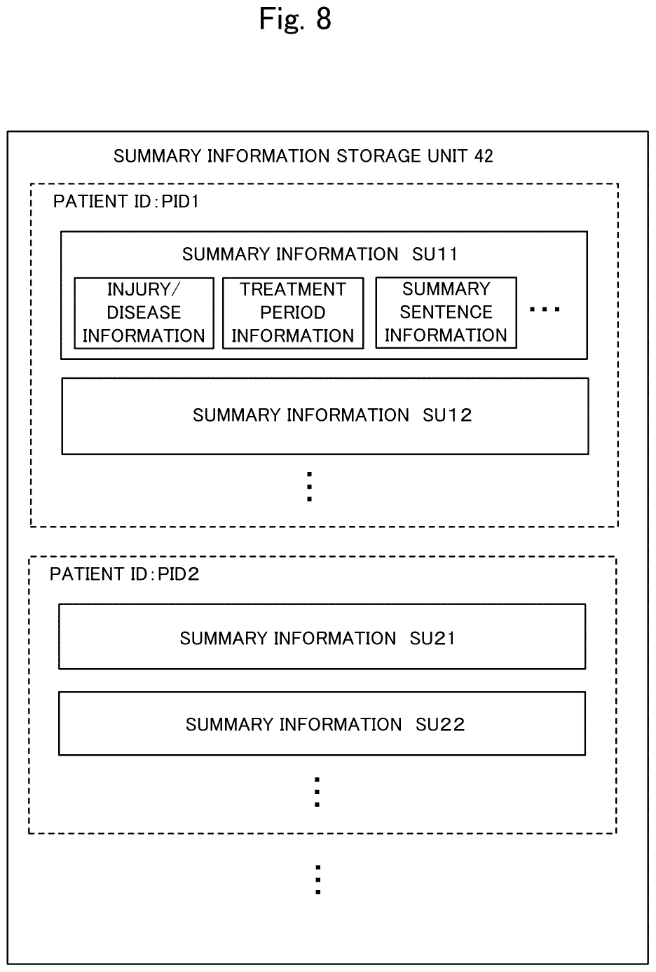

FIG. 8 is an example of a data structure of the summary information in the summary information storage unit 42. The summary information storage unit 42 stores, for example, the summary information indicating a summary of medical treatment records created for each treatment period in association with a patient ID for identifying a patient who has received a medical treatment. In the example of FIG. 8, the summary information storage unit 42 stores summary information “SU11”, summary information“SU12”, and the like related to a patient indicated by a patient ID “PID1” in association with the patient ID “PID1”, and stores summary information “SU21”, summary information “SU22”, and the like related to a patient indicated by a patient ID “PID2” in association with the patient ID “PID2”.

Each record of the summary information includes injury/disease information indicating injury/disease subjected to a treatment, treatment period information indicating a treatment period of the injury/disease, and summary sentence information indicating summary sentences obtained by summarizing medical treatment records in the treatment period. The summary sentence information includes a plurality of summary sentences according to a plurality of candidates for a summarization degree that may be set by a health information processing device 1A. The summary information may be generated by the health information processing device 1A or may be generated by a device other than the health information processing device 1A.

The health information processing device 1A has, for example, the same hardware configuration and functional blocks as those of the health information processing device 1 according to the first example embodiment. When generating a first medical treatment summary, the health information processing device 1A generates the first medical treatment summary with reference to the summary information storage unit 42. Specifically, a processor 11 of the health information processing device 1A has, for example, the functional blocks illustrated in FIG. 4, and a summary generation unit 17 in the second example embodiment refers to, from the summary information storage unit 42, summary information related to an object patient and treatment period related to the first medical summary to be generated in a case where the first medical treatment summary is generated based on a summarization degree set by a summarization degree setting unit 16. The summary generation unit 17 then acquires summary sentences related to the summarization degree set by the summarization degree setting unit 16 from summary sentence information of the summary information to be referred to, and generates the first medical treatment summary indicating the acquired summary sentences.

When the summarization degree is calculated, the summarization degree setting unit 16 in the second example embodiment may specify injury/disease in each treatment period with reference to the summary information storage unit 42 instead of specifying the injury/disease in each treatment period based on target medical treatment records supplied from a target medical treatment record acquisition unit 15. In this case, the summarization degree setting unit 16 acquires related summary information from the summary information storage unit 42 based on an object patient and a target period notified from the target medical treatment record acquisition unit 15, and specifies injury/disease based on injury/disease information of the summary information. In this case, the target medical treatment record acquisition unit 15 does not have to perform processing of acquiring target medical treatment records from the medical treatment record storage unit 41. That is, the storage device 4A in the second example embodiment does not necessarily include the medical treatment record storage unit 41.

As described above, the health information processing device 1A in the second example embodiment can generate a second medical treatment summary based on the summary information stored in the summary information storage unit 42.

### Third Example Embodiment

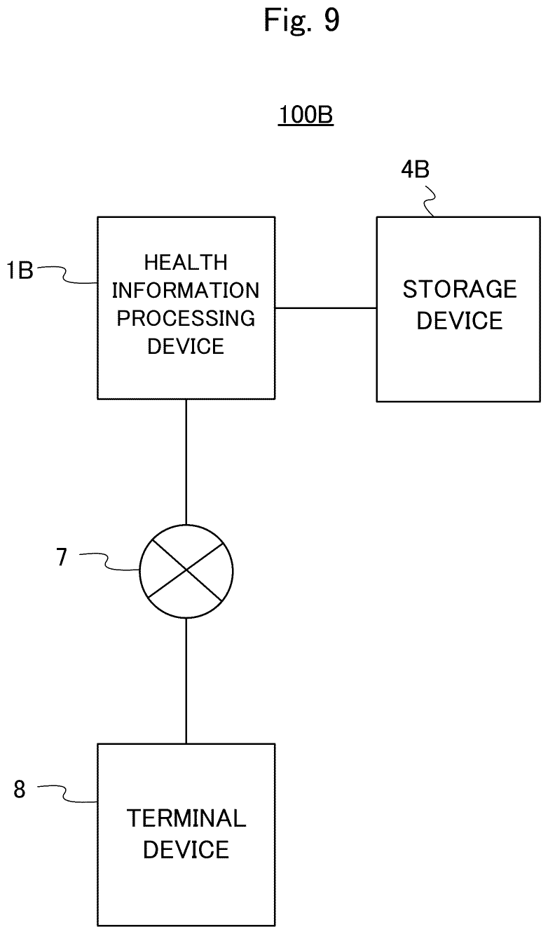

FIG. 9 illustrates a schematic configuration of a health information processing system 100B in a third example embodiment. The health information processing system 100B according to the third example embodiment is a server client model system, and a health information processing device 1B functioning as a server device performs the processing of the health information processing device 1 in the first example embodiment or the health information processing device 1A in the second example embodiment. Hereinafter, the same components as those of the first example embodiment or the second example embodiment are appropriately denoted by the same reference signs, and description thereof will be omitted.

The health information processing system 100B mainly includes the health information processing device 1B that functions as a server, a storage device 4B that stores data similar to the data stored in the storage device 4 according to the first example embodiment or the storage device 4A according to the second example embodiment, and a terminal device 8 that functions as a client. The health information processing device 1B and the terminal device 8 perform data communication via a network 7.

The terminal device 8 is a terminal having an input function, a display function, and a communication function, and functions as the input device 2 and the output device 3 illustrated in FIG. 1. The terminal device 8 may be, for example, a personal computer, a tablet terminal, a personal digital assistant (PDA), or the like. The terminal device 8 transmits an input signal or the like based on a user input to the health information processing device 1B.

The health information processing device 1B has the same hardware configuration and functional block configuration as those of the health information processing device 1 according to the first example embodiment or the health information processing device 1A according to the second example embodiment. The health information processing device 1B then receives the input signal and the like from the terminal device 8 via the network 7, specifies an object patient, a target period, and the like based on the received information, and generates a related second medical treatment summary, and the like. The health information processing device 1B exchanges an output signal indicating information or the like related to a generation result of the second medical treatment summary and an input signal indicating information or the like related to correction of the generated second medical treatment summary with the terminal device 8 via the network 7. That is, in this case, the terminal device 8 functions as the input device 2 and the output device 3 in the first example embodiment or the second example embodiment.

As described above, the health information processing system 100B in the third example embodiment can generate a second medical treatment summary related to an object patient and a target period specified by a user and present the second medical treatment summary to the user.

### Fourth Example Embodiment

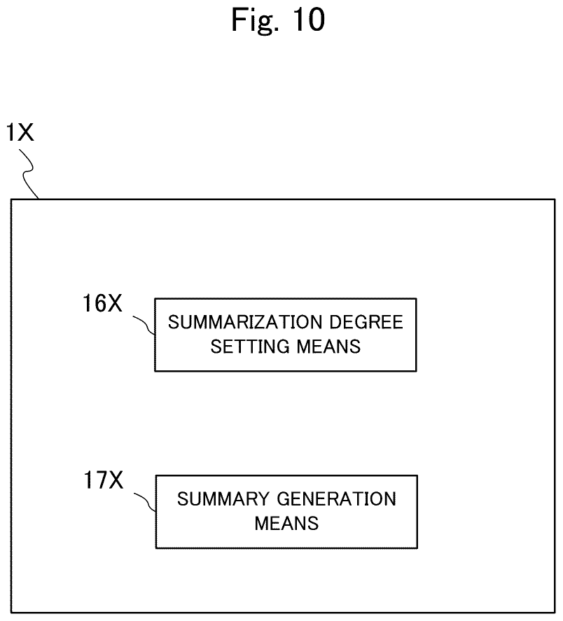

FIG. 10 is a block diagram of a health information processing device 1X according to the fourth example embodiment. The health information processing device 1X mainly includes a summarization degree setting means 16X and a summary generation means 17X. The health information processing device 1X may be configured by a plurality of devices.

The summarization degree setting means 16X is configured to set, based on a relationship of injury/disease between a plurality of treatment periods of a subject, a degree of summarization of medical treatment records for each of the plurality of treatment periods. Examples of the summarization degree setting means 16X include the summarization degree setting unit 16 according to any of the first to third example embodiments.

The summary generation means 17X is configured to generate a summary of the medical treatment records in a target period including the plurality of treatment periods based on the medical treatment records and the degree of summarization. Examples of the summary generation means 17X include the summary generation unit 17 according to any of the first to third example embodiments.

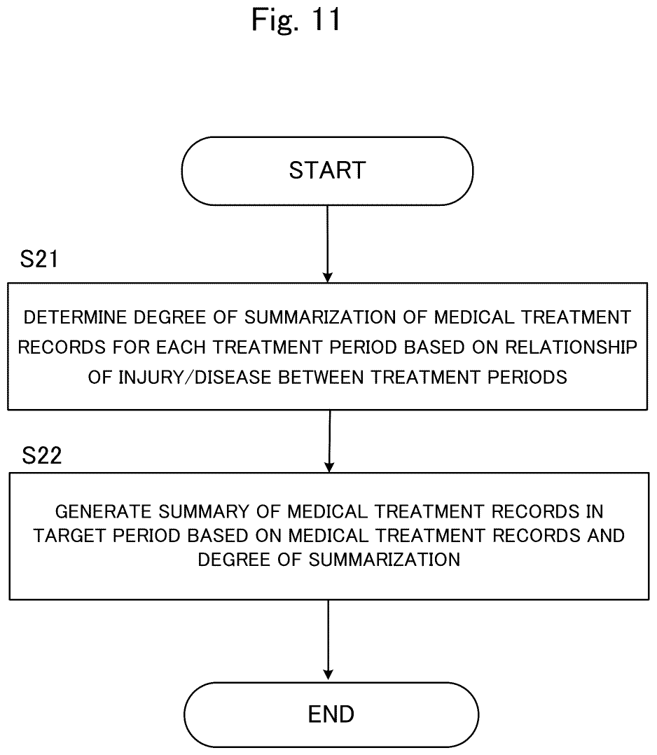

FIG. 11 illustrates an example of the flowchart executed by the health information processing device 1X according to the fourth example embodiment. The summarization degree setting means 16X is configured to set, based on a relationship of injury/disease between a plurality of treatment periods of a subject, a degree of summarization of medical treatment records for each of the plurality of treatment periods (step S21). The summary generation means 17X is configured to generate a summary of the medical treatment records in a target period including the plurality of treatment periods based on the medical treatment records and the degree of summarization (step S22).

According to the fourth example embodiment, the health information processing device 1X can generate the summary of medical treatment records in a target period including a plurality of treatment periods based on the relationship of injury/disease between the plurality of treatment periods.

In the example embodiments described above, the program is stored by any type of a non-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

The whole or a part of the example embodiments described above (including modifications, the same applies hereinafter) can be described as, but not limited to, the following Supplementary Notes.

### [Supplementary Note 1]

A health information processing device comprising:
a summarization degree setting means for setting, based on a relationship of injury/disease between a plurality of treatment periods of a subject, a degree of summarization of medical treatment records for each of the plurality of treatment periods; and
a summary generation means for generating a summary of the medical treatment records in a target period including the plurality of treatment periods based on the medical treatment records and the degree of summarization.

### [Supplementary Note 2]

The health information processing device according to Supplementary Note 1, wherein the summarization degree setting means sets the degree of summarization for each of the plurality of treatment periods based on a relationship between injury/disease in a last treatment period in the target period and injury/disease in each of the plurality of treatment periods.

### [Supplementary Note 3]

The health information processing device according to Supplementary Note 2, wherein, as a treatment period has a higher degree of relevance of injury/disease to the last treatment period in the target period, the summarization degree setting means sets the degree of summarization in the treatment period to be lower.

### [Supplementary Note 4]

The health information processing device according to Supplementary Note 1, wherein the summarization degree setting means specifies the injury/disease for each of the plurality of treatment periods based on the medical treatment records for each of the plurality of treatment periods.

### [Supplementary Note 5]

The health information processing device according to Supplementary Note 1, wherein
the summary generation means acquires a first medical treatment summary obtained by summarizing the medical treatment records related to each of the plurality of treatment periods by the degree of summarization related to each of the plurality of treatment periods, and
generates a second medical treatment summary that is a summary of the medical treatment records in the target period based on the first medical treatment summary related to each of the plurality of treatment periods.

### [Supplementary Note 6]

The health information processing device according to Supplementary Note 5, further comprising
a medical treatment record acquisition means for acquiring the medical treatment records for each of the plurality of treatment periods,
wherein the summary generation means generates the first medical treatment summary for each of the plurality of treatment periods based on the medical treatment records for each of the plurality of treatment periods and the degree of summarization related to each of the plurality of treatment periods.

### [Supplementary Note 7]

The health information processing device according to Supplementary Note 5, wherein the summary generation means acquires the first medical treatment summary with reference to summary information indicating a relationship between the degree of summarization and summary sentences obtained by summarizing the medical treatment records related to each of the plurality of treatment periods based on the degree of summarization.

### [Supplementary Note 8]

The health information processing device according to Supplementary Note 1, further comprising a correction input reception means for receiving a correction input of the summary in the target period.

### [Supplementary Note 9]

The health information processing device according to Supplementary Note 1, further comprising a correction means for correcting the summary in the target period in such a way as to fall within an entry frame of a medical document to be applied.

### [Supplementary Note 10]

A health information processing method by a computer, the health information processing method comprising:
setting, based on a relationship of injury/disease between a plurality of treatment periods of a subject, a degree of summarization of medical treatment records for each of the plurality of treatment periods; and
generating a summary of the medical treatment records in a target period including the plurality of treatment periods based on the medical treatment records and the degree of summarization.

### [Supplementary Note 11]

A storage medium storing a program for causing a computer to execute processing comprising:
setting, based on a relationship of injury/disease between a plurality of treatment periods of a subject, a degree of summarization of medical treatment records for each of the plurality of treatment periods; and
generating a summary of the medical treatment records in a target period including the plurality of treatment periods based on the medical treatment records and the degree of summarization.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. All Patent and Non-Patent Literatures mentioned in this specification are incorporated by reference in its entirety.

### DESCRIPTION OF REFERENCE NUMERALS

1, 1A, 1B, 1X Health information processing device
2 Input device
3 Output device
4 Storage device
8 Terminal device
100, 100A, 100B Health information processing system

What is claimed is:

1. A health information processing device comprising:
at least one memory configured to store instructions; and
at least one processor configured to execute the instructions to:
set, based on a relationship of injury/disease between a plurality of treatment periods of a subject, a degree of summarization of medical treatment records for each of the plurality of treatment periods; and
generate a summary of the medical treatment records in a target period including the plurality of treatment periods based on the medical treatment records and the degree of summarization.

2. The health information processing device according to 1, wherein the at least one processor is configured to execute the instructions to set the degree of summarization for each of the plurality of treatment periods based on a relationship between injury/disease in a last treatment period in the target period and injury/disease in each of the plurality of treatment periods.

3. The health information processing device according to claim 2, wherein, as a treatment period has a higher degree of relevance of injury/disease to the last treatment period in the target period, the at least one processor is configured to execute the instructions to set the degree of summarization in the treatment period to be lower.

4. The health information processing device according to claim 1, wherein the at least one processor is configured to execute the instructions to specify the injury/disease for each of the plurality of treatment periods based on the medical treatment records for each of the plurality of treatment periods.

5. The health information processing device according to claim 1, wherein
the at least one processor is configured to execute the instructions to
acquire a first medical treatment summary obtained by summarizing the medical treatment records related to each of the plurality of treatment periods by the degree of summarization related to each of the plurality of treatment periods, and
generate a second medical treatment summary that is a summary of the medical treatment records in the target period based on the first medical treatment summary related to each of the plurality of treatment periods.

6. The health information processing device according to claim 5,
wherein the at least one processor is configured to execute the instructions to acquire the medical treatment records for each of the plurality of treatment periods,
wherein the at least one processor is configured to execute the instructions to generate the first medical treatment summary for each of the plurality of treatment periods based on the medical treatment records for each of the plurality of treatment periods and the degree of summarization related to each of the plurality of treatment periods.

7. The health information processing device according to claim 5, wherein the at least one processor is configured to execute the instructions to acquire the first medical treatment summary with reference to summary information indicating a relationship between the degree of summarization and summary sentences obtained by summarizing the medical treatment records related to each of the plurality of treatment periods based on the degree of summarization.

8. The health information processing device according to claim 1,
wherein the at least one processor is configured to execute the instructions to receive a correction input of the summary in the target period.

9. The health information processing device according to claim 1,
wherein the at least one processor is configured to execute the instructions to correct the summary in the target period in such a way as to fall within an entry frame of a medical document to be applied.

10. A health information processing method by a computer, the health information processing method comprising:
setting, based on a relationship of injury/disease between a plurality of treatment periods of a subject, a degree of summarization of medical treatment records for each of the plurality of treatment periods; and
generating a summary of the medical treatment records in a target period including the plurality of treatment periods based on the medical treatment records and the degree of summarization.

11. A non-transitory computer readable storage medium storing a program for causing a computer to execute processing comprising:
setting, based on a relationship of injury/disease between a plurality of treatment periods of a subject, a degree of summarization of medical treatment records for each of the plurality of treatment periods; and
generating a summary of the medical treatment records in a target period including the plurality of treatment periods based on the medical treatment records and the degree of summarization.
